SEQUENCE LISTING (60 sequences)

SEQ ID NO: 1 - DNA, 6 bp, Artificial Sequence
  Synthetic polynucleotide
  misc_feature (1)..(2): n is a, c, g, or t
nngrrt

SEQ ID NO: 2 - DNA, 5 bp, Artificial Sequence
  Synthetic polynucleotide
  misc_feature (1)..(2): n is a, c, g, or t
nngrr

SEQ ID NO: 3 - DNA, 20 bp, Artificial Sequence
  Synthetic polynucleotide
gcgcgctcgctcgctcactg

SEQ ID NO: 4 - DNA, 20 bp, Artificial Sequence
  Synthetic polynucleotide
gctcgctcactgaggccgcc

SEQ ID NO: 5 - DNA, 19 bp, Artificial Sequence
  Synthetic polynucleotide
ctcgctcactgaggcgccc

SEQ ID NO: 6 - DNA, 20 bp, Artificial Sequence
  Synthetic polynucleotide
acgcccgggctttgcccggg

SEQ ID NO: 7 - DNA, 20 bp, Artificial Sequence
  Synthetic polynucleotide
ccgacgcccgggctttgccc

SEQ ID NO: 8 - DNA, 20 bp, Artificial Sequence
  Synthetic polynucleotide
cccgacgcccgggctttgcc

SEQ ID NO: 9 - DNA, 20 bp, Artificial Sequence
  Synthetic polynucleotide
gaggccgcccgggcaaagcc

SEQ ID NO: 10 - DNA, 20 bp, Artificial Sequence
  Synthetic polynucleotide
aggccgcccgggcaaagccc

SEQ ID NO: 11 - DNA, 20 bp, Artificial Sequence
  Synthetic polynucleotide
cccgggcaaagcccgggcgt

SEQ ID NO: 12 - DNA, 20 bp, Artificial Sequence
  Synthetic polynucleotide
ccaaaggtcgcccgacgccc

SEQ ID NO: 13 - DNA, 20 bp, Artificial Sequence
  Synthetic polynucleotide
ccgggcaaagcccgggcgtc

SEQ ID NO: 14 - DNA, 20 bp, Artificial Sequence
  Synthetic polynucleotide
accaaaggtcgcccgacgcc

SEQ ID NO: 15 - DNA, 20 bp, Artificial Sequence
  Synthetic polynucleotide
cccgggcgtcgggcgacctt

SEQ ID NO: 16 - DNA, 19 bp, Artificial Sequence
  Synthetic polynucleotide
cactaggccgggcgaccaa

SEQ ID NO: 17 - DNA, 20 bp, Artificial Sequence
  Synthetic polynucleotide
tcgggcgacctttggtcgcc

SEQ ID NO: 18 - DNA, 20 bp, Artificial Sequence
  Synthetic polynucleotide
ctcgctcgctcactgaggcc

SEQ ID NO: 19 - DNA, 20 bp, Artificial Sequence
  Synthetic polynucleotide
gctcgctcgctcactgaggc

SEQ ID NO: 20 - DNA, 20 bp, Artificial Sequence
  Synthetic polynucleotide
gcgcgctcgctcgctcactg

SEQ ID NO: 21 - DNA, 20 bp, Artificial Sequence
  Synthetic polynucleotide
gagcgagcgagcgcgcagag

SEQ ID NO: 22 - DNA, 20 bp, Artificial Sequence
  Synthetic polynucleotide
agcgagcgagcgcgcagaga

SEQ ID NO: 23 - DNA, 20 bp, Artificial Sequence
  Synthetic polynucleotide
gcgagcgcgcagagagggag

SEQ ID NO: 24 - DNA, 20 bp, Artificial Sequence
  Synthetic polynucleotide
ggaacccctagtgatggagt

SEQ ID NO: 25 - DNA, 20 bp, Artificial Sequence
  Synthetic polynucleotide
gagtggccaactccatcact

SEQ ID NO: 26 - DNA, 20 bp, Artificial Sequence
  Synthetic polynucleotide
agtggccaactccatcacta

SEQ ID NO: 27 - DNA, 20 bp, Artificial Sequence
  Synthetic polynucleotide
ctacaaggaacccctagtga

SEQ ID NO: 28 - DNA, 20 bp, Artificial Sequence
  Synthetic polynucleotide
gtggccaactccatcactag

SEQ ID NO: 29 - DNA, 21 bp, Artificial Sequence
  Synthetic polynucleotide
gagcgagcgagcgcgcagaga

SEQ ID NO: 30 - DNA, 21 bp, Artificial Sequence
  Synthetic polynucleotide
ggagtggccaactccatcact

SEQ ID NO: 31 - DNA, 21 bp, Artificial Sequence
  Synthetic polynucleotide
actacaaggaacccctagtga

SEQ ID NO: 32 - DNA, 3 bp, Artificial Sequence
  Synthetic polynucleotide
agg

SEQ ID NO: 33 - DNA, 3 bp, Artificial Sequence
  Synthetic polynucleotide
cgg

SEQ ID NO: 34 - DNA, 3 bp, Artificial Sequence
  Synthetic polynucleotide
ggg

SEQ ID NO: 35 - DNA, 3 bp, Artificial Sequence
  Synthetic polynucleotide
cgg

SEQ ID NO: 36 - DNA, 3 bp, Artificial Sequence
  Synthetic polynucleotide
ggg

SEQ ID NO: 37 - DNA, 3 bp, Artificial Sequence
  Synthetic polynucleotide
cgg

SEQ ID NO: 38 - DNA, 3 bp, Artificial Sequence
  Synthetic polynucleotide
cgg

SEQ ID NO: 39 - DNA, 3 bp, Artificial Sequence
  Synthetic polynucleotide
ggg

SEQ ID NO: 40 - DNA, 3 bp, Artificial Sequence
  Synthetic polynucleotide
cgt

SEQ ID NO: 41 - DNA, 3 bp, Artificial Sequence
  Synthetic polynucleotide
ggg

SEQ ID NO: 42 - DNA, 3 bp, Artificial Sequence
  Synthetic polynucleotide
ggg

SEQ ID NO: 43 - DNA, 3 bp, Artificial Sequence
  Synthetic polynucleotide
cgg

SEQ ID NO: 44 - DNA, 3 bp, Artificial Sequence
  Synthetic polynucleotide
tgg

SEQ ID NO: 45 - DNA, 3 bp, Artificial Sequence
  Synthetic polynucleotide
agg

SEQ ID NO: 46 - DNA, 3 bp, Artificial Sequence
  Synthetic polynucleotide
cgg

SEQ ID NO: 47 - DNA, 3 bp, Artificial Sequence
  Synthetic polynucleotide
ggg

SEQ ID NO: 48 - DNA, 3 bp, Artificial Sequence
  Synthetic polynucleotide
cgg

SEQ ID NO: 49 - DNA, 3 bp, Artificial Sequence
  Synthetic polynucleotide
agg

SEQ ID NO: 50 - DNA, 3 bp, Artificial Sequence
  Synthetic polynucleotide
agg

SEQ ID NO: 51 - DNA, 3 bp, Artificial Sequence
  Synthetic polynucleotide
ggg

SEQ ID NO: 52 - DNA, 3 bp, Artificial Sequence
  Synthetic polynucleotide
tgg

SEQ ID NO: 53 - DNA, 3 bp, Artificial Sequence
  Synthetic polynucleotide
tgg

SEQ ID NO: 54 - DNA, 3 bp, Artificial Sequence
  Synthetic polynucleotide
agg

SEQ ID NO: 55 - DNA, 3 bp, Artificial Sequence
  Synthetic polynucleotide
ggg

SEQ ID NO: 56 - DNA, 3 bp, Artificial Sequence
  Synthetic polynucleotide
tgg

SEQ ID NO: 57 - DNA, 3 bp, Artificial Sequence
  Synthetic polynucleotide
ggg

SEQ ID NO: 58 - DNA, 6 bp, Artificial Sequence
  Synthetic polynucleotide
gggagt

SEQ ID NO: 59 - DNA, 6 bp, Artificial Sequence
  Synthetic polynucleotide
aggggt

SEQ ID NO: 60 - DNA, 6 bp, Artificial Sequence
  Synthetic polynucleotide
tggagt

(19) United States
(12) Patent Application Publication
(10) Pub. No.: US 2021/0261982 A1
(43) Pub. Date: Aug. 26, 2021

(54) RAAV-MEDIATED NUCLEASE-ASSOCIATED VECTOR INTEGRATION (RAAV-NAVI)

(71) Applicant: University of Massachusetts, Boston, MA (US)

(72) Inventors: Alexander Brown, Worcester, MA (US); Guangping Gao, Westborough, MA (US)

(73) Assignee: University of Massachusetts, Boston, MA (US)

(21) Appl. No.: 17/051,197

(22) PCT Filed: Apr. 29, 2019
(86) PCT No.: PCT/US2019/029659

§ 371 Date: Oct. 28, 2020

Related U.S. Application Data
(60) Provisional application No. 62/664,198, filed on Apr. 29, 2018.

(51) Int. Cl. C12N 15/86; C12N 9/22; C12N 15/11

(52) U.S. Cl. CPC C12N 15/86; C12N 15/111; C12N 9/22

(57) ABSTRACT

Aspects of the disclosure relate to integration of a transgene packaged into recombinant adeno-associated virus (rAAV) by nuclease-assisted vector integration (NAVI).

### RELATED APPLICATION

This Application is a national stage filing under 35 U.S.C. § 371 of international PCT application, PCT/US2019/029659, filed Apr. 29, 2019, which claims priority under 35 U.S.C. § 119(e) to U.S. provisional patent application, U.S. Ser. No. 62/664,198, filed Apr. 29, 2018, the entire contents of each of which are incorporated herein by reference.

### BACKGROUND

Previously described methods for site-specific therapeutic transgene integration in vivo lack precision, efficiency, and long-term stability. Nuclease-assisted vector integration (NAVI) has been used successfully for in vitro gene editing. NAVI relies on non-homologous end joining (NHEJ) pathways to insert a double-stranded DNA template vector at a target gene following cleavage of the target gene by engineered nucleases. However, previous attempts to adapt NAVI for in vivo gene editing have been unsuccessful, in large part because of a previous lack of understanding regarding how to engineer the system to limit inclusion of viral elements within the host cell genome.

### SUMMARY

Aspects of the disclosure relate to integration of a transgene packaged into recombinant adeno-associated virus (rAAV) by nuclease-assisted vector integration (NAVI). In some embodiments, the safety of rAAV transgene integration is enhanced utilizing guide RNAs (gRNAs) that remove viral AAV inverted terminal repeats (ITRs) prior to host genome integration.

Accordingly, in some aspects, the disclosure provides an isolated nucleic acid comprising at least one transgene flanked by adeno-associated virus (AAV) inverted terminal repeats (ITRs). In some embodiments, the transgene is configured to be integrated into a target genome by nuclease-assisted vector integration (NAVI). In some embodiments, the guide RNAs are configured to direct removal (e.g., cleavage) of the ITR sequences, e.g., prior to transgene integration.

In some aspects, the disclosure provides an isolated nucleic acid comprising an expression cassette engineered to express a first guide RNA (gRNA) flanked by AAV inverted terminal repeats (ITRs). In some embodiments, the gRNA targets (e.g., hybridizes with) a nucleic acid sequence located within the nucleic acid sequence encoding the ITRs.

In some embodiments, a gRNA comprises a NNGRRT (SEQ ID NO: 1) or a NNGRR (SEQ ID NO: 2) sequence. In some embodiments, a gRNA comprises a sequence set forth in Table 1.

In some embodiments, the expression cassette is further engineered to express a second gRNA that targets (e.g. hybridizes with) a target nucleic acid sequence that is not present in the isolated nucleic acid.

In some embodiments, a target nucleic acid sequence is located in a host cell (e.g., a mammalian cell, such as a human cell).

In some embodiments, a target nucleic acid sequence is present in a safe harbor genome locus. In some embodiments, a safe harbor genome locus is AAVS1 genome locus.

In some embodiments, the expression cassette is further engineered to express an mRNA that encodes a protein. In some embodiments, a protein is a reporter protein or a therapeutic protein.

In some aspects, the disclosure provides a recombinant adeno-associated virus (rAAV) comprising: an isolated nucleic acid as described by the disclosure; and at least one AAV capsid protein.

In some embodiments, at least one capsid protein is AAV1, AAV2, AAV3, AAV4, AAV5, AAV6, AAV7, AAV8, AAV9 capsid protein. In some embodiments, at least one capsid protein is an AAV9 capsid protein.

In some aspects, the disclosure provides a composition comprising: an rAAV as described by the disclosure; and a nuclease.

In some embodiments, a nuclease is a Transcription Activator-like Effector Nuclease (TALEN), Zinc-Finger Nuclease (ZFN), engineered meganuclease, re-engineered homing endonuclease, or a Cas-family nuclease. In some embodiments, a nuclease is a Cas-family nuclease. In some embodiments, a Cas-family nuclease is a Cas9 or Cas7 nuclease, for example a Streptococcus pyogenes (Sp) or a Staphylococcus aureus (Sa) Cas9 nuclease. In some embodiments, a nuclease is encoded by a plasmid or a viral vector. In some embodiments, a viral vector is an rAAV vector.

In some aspects, the disclosure provides methods for inserting a gene into a target locus of a genome, the methods comprising introducing into a cell: an isolated nucleic acid or rAAV as described herein, and a nuclease. In some aspects, the disclosure provides methods for inserting a gene into a target locus of a genome, the methods comprising introducing into a cell a composition as described by the disclosure.

In some embodiments of methods described by the disclosure, introducing an isolated nucleic acid and a nuclease into a cell results in insertion of the transgene encoded by the viral vector into a target locus without any viral nucleic acid sequence (e.g., AAV ITR sequence) being inserted.

some embodiments, a target locus is a safe harbor genome locus, for example an AAVS1 genome locus.

In some embodiments, a cell is in a subject. In some embodiments, a subject is a mammal, such as a human. In some embodiments, a subject has or is suspected of having a disease.

In some embodiments, a cell is in vitro or ex vivo.

In some embodiments, a cell is characterized by aberrant expression (e.g., over-expression or reduced expression relative to a normal cell) or aberrant function (e.g., increased activity or reduced activity relative to a normal cell), of a protein.

### BRIEF DESCRIPTION OF DRAWINGS

FIGS. 1A-1E show rAAV-mediated NAVI design and detection. FIG. 1A shows the rAAV vector design and integration strategy. FIG. 1B shows probe (left) and traditional primer (right) configurations for the detection and quantification of plus (top) and minus (bottom) vector integration patterns within genomic safe harbor by PCR amplification. FIG. 1C shows a representative end-point PCR detection of vector integration from mouse liver tissue 4 weeks after neonatal infection with rAAV-NAVI virus (10¹¹ viral copies/pup, facial vein) with preferential vector orientation. Analyses of heart (FIG. 1D) and muscle (FIG. 1E) genomic DNA indicate tissue-specific patterns of integration achieved by rAAV-NAVI.

FIGS. 2A-2F show quantification of rAAV-NAVI transgene expression in liver by fluorescence microscopy following neonatal injection 4-weeks post-infection. FIG. 2A shows percentage of cells positive for mCherry in NAVI and control (rAAV) groups. FIG. 2B shows relative intensity of mCherry in NAVI and control (rAAV) groups. FIG. 2C shows mCherry intensity in positive NAVI and control (rAAV) cells. Tissues were also analyzed from mice that underwent partial hepatectomy at 3-months post-infection, followed by 4-week recovery (FIGS. 2D-2F).

FIGS. 3A-3B show representative fluorescence microscopy images of tissues obtained pre- (FIG. 3A) and post- (FIG. 3B) hepatectomy. Cell nuclei are stained with DAPI and transgene expression was detected by fluorescence of the mCherry reporter.

### DETAILED DESCRIPTION OF INVENTION

Aspects of the disclosure relate to integration of a transgene packaged into recombinant adeno-associated virus (rAAV) by nuclease-assisted vector integration (NAVI). In some embodiments, the safety and efficacy of the integration of the transgene is enhanced through the use of guide RNAs (gRNAs) that remove viral AAV inverted terminal repeats (ITRs) prior to integration into the host genome. Using the compositions and methods described herein, the transgene can be integrated without the ITR elements or additional, unintended vector cleavage fragments. Further, in some embodiments, methods described herein utilize target nucleic acid sequence that are located in a safe harbor genome loci distinct from genomic coding sequences.

Aspects of AAV-NAVI are based upon non-homologous end joining (NHEJ) pathways gene editing of a transgene (e.g., to delete or remove the ITRs) and gene editing of a nucleic acid sequence in the host genome using engineered nucleases to achieve homology-independent targeted integration of the transgene into genomic DNA. The efficiency of gene editing and flexibility in target nucleic acid selection by this approach are typically higher than homology-directed repair (HDR) methods, and therefore, may facilitate the genetic modification of cells that are otherwise resistant to editing by HDR (e.g., post-mitotic cells). Targeted gene editing using AAV-NAVI is initiated when a vector is co-delivered with nucleases, e.g., TALENs or Cas9 endonucleases, and appropriate guide RNAs (or introduced into a cell containing one or more of the foregoing components),thereby inducing a double-strand break (DSB) at the target genomic locus and in the transfer vector(s). Since the genomic DSB and vector linearization are linked spatially and temporally by the co-delivered nuclease, vector integration at the genomic DSB by endogenous non-homologous end joining (NHEJ) repair pathways occurs.

The genome of rAAV encoding a transgene may be either single-stranded (ss) or self-complimentary (sc) DNA, flanked at either end by inverted terminal repeats (ITR) elements that are necessary for packaging into the viral capsid.

The disclosure is based, in part, of NAVI-AAV constructs engineered to limit inclusion of viral elements within a host cell genome. In some embodiments, the disclosure provides rAAVs adapted for NAVI, which initiate vector cleavage at sites within or proximal to the ITRs of the rAAV. In this manner, the entire rAAV genome is integrated into a host cell genome without the ITR elements or additional, unintended vector cleavage fragments. In some embodiments, NAVI-AAV is targeted to genomic safe harbor loci, which encourages stable integration by eliminating the re-formation of target sites following vector integration. In some embodiments, a single guide RNA strategy is be adapted through cloning of the genomic target sites on either end of the transgenomic DNA.

### Gene Editing

Methods of gene editing using AAV-NAVI, e.g., to insert a gene into a target locus of a genome, are provided by the disclosure. The methods typically involve exposing a cell to an isolated nucleic acid or recombinant adeno-associated viral (rAAV) vector as described herein and a nuclease. In some embodiments, an isolated nucleic acid comprises at least one transgene flanked by inverted terminal repeats (ITRs), wherein the transgene is configured to be integrated into a target genome by nuclease-assisted vector integration, such that guide RNAs direct removal of the ITRs prior to transgene integration. In some embodiments, an isolated nucleic acid comprises an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs.

As used herein, “gene editing” refers to adding, disrupting or changing genomic sequences (e.g., a gene sequence) and is performed using gene editing molecules such as engineered nucleases and/or nucleic acids, e.g., guide RNAs. In some aspects, gene editing comprises the use of engineered nucleases to cleave a target genomic locus. In some embodiments, gene editing further comprises inserting, deleting, mutating or substituting nucleic acid residues at a cleaved locus. In some embodiments, inserting, deleting, mutating or substituting nucleic acid residues at a cleaved locus is accomplished through endogenous non-homologous end joining (NHEJ) repair pathways.

As used herein, the term a “gene editing molecule” refers to a molecule (e.g., nucleic acid or protein) capable of directing or affecting gene editing. Exemplary gene editing molecules include, but are not limited to, nucleases and recombinases, as well as nucleic acids that guide the activity of such enzymes, e.g., guide RNAs.

As used herein, the terms “endonuclease” and “nuclease” refer to an enzyme that cleaves a phosphodiester bond or bonds within a polynucleotide chain. Nucleases may be naturally occurring or genetically engineered. Genetically engineered nucleases are particularly useful for gene editing and are generally classified into four families: zinc finger nucleases (ZFNs), transcription activator-like effector nucleases (TALENs), engineered meganucleases and CRISPR-associated proteins (Cas nucleases). In some embodiments, the nuclease is a Transcription Activator-like Effector Nuclease (TALEN), a Zinc-Finger Nuclease (ZFN), an engineered meganuclease, a re-engineered homing endonuclease, or a Cas-family nuclease. In some embodiments, the nuclease is a ZFN. In some embodiments, the ZFN comprises a Fokl cleavage domain. In some embodiments, the ZFN comprises Cys2His2 fold group. In some embodiments, the nuclease is a TALEN. In some embodiments, the TALEN comprises a Fokl cleavage domain. In some embodiments, the nuclease is an engineered meganuclease.

The term “CRISPR” refers to “clustered regularly interspaced short palindromic repeats,” which are DNA loci containing short repetitions of base sequences. CRISPR loci form a portion of a prokaryotic adaptive immune system that confers resistance to foreign genetic material. Each CRISPR loci is flanked by short segments of “spacer DNA,” which are derived from viral genomic material. In the Type II CRISPR system, spacer DNA hybridizes to transactivating RNA (tracrRNA) and is processed into CRISPR-RNA (crRNA) and subsequently associates with CRISPR-associated nucleases (Cas nucleases) to form complexes that recognize and degrade foreign DNA. In certain embodiments, the nuclease is a CRISPR-associated nuclease (Cas nuclease).

For the purpose of gene editing, the CRISPR system can be modified to combine the tracrRNA and crRNA in to a single guide RNA (sgRNA) or just (gRNA). As used herein, the term “guide RNA” or “gRNA” refers to a polynucleotide sequence that is complementary to a target sequence in a cell and associates with a Cas nuclease, thereby directing the Cas nuclease to the target sequence. In some embodiments, a sgRNA or gRNA ranges between 1 and 30 nucleotides in length. In some embodiments, a sgRNA or gRNA ranges between 5 and 25 nucleotides in length. In some embodiments, a sgRNA or gRNA ranges between 10 and 20 nucleotides in length. In some embodiments, a sgRNA or gRNA ranges between 14 and 18 nucleotides in length. In some embodiments, a sgRNA or gRNA ranges between 5 and 50 nucleotides in length. In some embodiments, a sgRNA or gRNA is 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, a sgRNA can comprise a spacer sequence, a minimum CRISPR repeat sequence, a linker, a minimum tracrRNA sequence, a 3′ tracrRNA sequence. In some embodiments, a sgRNA may further comprise a spacer extension sequence and/or a tracrRNA extension sequence.

In some embodiments, a sgRNA or gRNA targets (e.g., hybridizes with) a nucleic acid sequence located adjacent to or within a nucleic acid sequence encoding an ITR of an isolated nucleic acid. In some embodiments, a gRNA targets a nucleic acid adjacent to an ITR at the 5′ or 3′ end of the ITR. In some embodiments, the gRNA comprises a NNGRRT (SEQ ID NO: 1) sequence, optionally wherein N is any nucleotide and R is A or G. In some embodiments, the gRNA comprises a NNGRR (SEQ ID NO: 2) sequence, optionally wherein N is any nucleotide and R is A or G. In some embodiments, the gRNA comprises any one of the sequences set forth in Table 1.

In some embodiments, a sgRNA or gRNA targets (e.g., hybridizes with) a target nucleic acid sequence that is not present in the isolated nucleic acid (e.g., sgRNA or gRNA does not target a nucleic acid sequence located adjacent to or within a nucleic acid sequence encoding an ITR of an isolated nucleic acid). In some embodiments, a gRNA targets a genomic sequence located in a host cell or subject. In some embodiments, a gRNA targets a genomic sequence located at a safe harbor locus in a host cell or subject.

In some embodiments, a first gRNA targets a nucleic acid sequence located adjacent to or within a nucleic acid sequence encoding an ITR of an isolated nucleic acid and a second gRNA targets a genomic nucleic acid sequence located in a host cell or subject.

In some embodiments, a gRNA is at least 75%, 80%, 85%, 90%, 95%, 97%, 99%, or 100% complementary to a nucleic acid sequence.

Examples of CRISPR nucleases include, but are not limited to Cas9, Cas6, Cas7, and Cpf1. In some embodiments, the nuclease is Cas9. In some embodiments, the Cas9 is a mutated Cas9. In some embodiments, the Cas9 is a truncated Cas9. In some embodiments, the Cas9 is derived from a bacteria. In some embodiments, the Cas9 is derived from the bacteria Streptococcus pyogenes (Sp). In some embodiments, the Cas9 is derived from the bacteria Staphylococcus aureus (Sa).

Recombinases are enzymes that mediate site-specific recombination by binding to nucleic acids via conserved recognition sites and mediating at least one of the following forms of DNA rearrangement: integration, excision/resolution and/or inversion. Recombinases are generally classified into two families of proteins, tyrosine recombinases and serine recombinases based on the active amino acid of the catalytic domain. Recombinases may further be classified according to their directionality (e.g., bidirectional or unidirectional). Bidirectional recombinases bind to identical recognition sites and therefore mediate reversible recombination. Non-limiting examples of identical recognition sites for bidirectional recombinases include loxP, FRT and RS recognition sites. Unidirectional recombinases bind to non-identical recognition sites and therefore mediate irreversible recombination.

In some embodiments, the disclosure relates to zinc finger nucleases. As used herein, a zinc finger nuclease (ZFN) refers to a protein which contains at least one structural motif characterized by the coordination of one or more zinc ions which stabilize the protein fold. Zinc fingers are among the most diverse structural motifs found in proteins, and up to 3% of human genes encode zinc fingers. Most ZFNs contain multiple zinc fingers which make tandem contacts with target molecules, including DNA, RNA, and the small protein ubiquitin. “Classical” zinc finger motifs are composed of 2 cysteine amino acids and 2 histidine amino acids (C₂H₂) and bind DNA in a sequence-specific manner. These ZFNs, which include transcription factor IIIIA (TFIIIA), are typically involved in gene expression. Multiple zinc finger motifs in DNA binding proteins bind and wrap around the outside of a DNA double helix. Due to their relatively small size (e.g., each finger is about 25-40, usually 27-35 amino acids), zinc finger nucleases are utilized to create DBDs with novel DNA binding specificity. These DBDs can deliver other fused domains (e.g., transcriptional activation or repression domains or epigenetic modification domains) to alter transcription regulation of a target gene. In some embodiments, zinc finger nucleases comprise 2 to 8 fingers, wherein each finger contains 27 to 40 amino acids (e.g., 27, 28, 29, 30 , 31 , 32, 33, 34, 35, 36, 37, 38, 39 or 40 amino acids).

In some embodiments, a ZFN comprises 1, 2, 3, 4, 5, 6, 7, or 8 zinc fingers. Each zinc finger may comprise 25-40, 25-30, 30-35, 35-40, or 40-45 amino acids. In some embodiments, a zinc finger comprises 27-35 amino acids. In some embodiments, a zinc finger comprises 27, 28, 29, 30, 31, 32, 33, 34, or 35 amino acids. A zinc finger may specifically recognize or bind to a target nucleic acid sequence. In some embodiments, a zinc finger comprises a recognition helix that recognizes or bind to a target nucleic acid sequence. In some embodiments, a recognition helix comprises 4-10 amino acids. In some embodiments, a recognition helix comprises 4, 6, 7, 8, 9, or 10 amino acids. In some embodiments, a zinc finger comprises a linker sequence at its C-terminal end that may serve to link or connect said zinc finger to an additional zinc finger. In some embodiments, a linker sequence may be a canonical linker on a non-canonical linker. In some embodiments, a linker sequence may be 2-10 amino acids, e.g., 2, 3, 4, 5, 6, 7, 8, 9, or 10 amino acids.

In some embodiments, nucleases are transcription activator-like effector nucleases (TALENs). A TALEN may specifically recognize or bind to a target nucleic acid sequence. Typically, a TALEN for use herein has been engineered to bind a target nucleic acid sequence through a central repeat domain consisting of a variable number of ˜30-35 amino acid repeats, wherein each repeat recognizes a single base pair within the target sequence. An array of these repeats are typically necessary to recognize a nucleic acid sequence.

In some embodiments, nucleases are homeodomains. A homeodomain may specifically recognize or bind to a target nucleic acid sequence. Homeodomains are proteins containing three alpha helices and an N-terminal arm that are responsible for recognizing a target sequence. A homeodomain typically recognizes a small DNA sequence (˜4 to 8 base pairs), however these domains can be fused in tandem with other DNA-binding domains (either other homeodomains or zinc finger proteins) to recognize longer extended sequences (12 to 24 base pairs).

### Isolated Nucleic Acid

In some aspects, the disclosure provides isolated nucleic acids that comprise at least one transgene flanked by inverted terminal repeats (ITRs), wherein the transgene is configured to be integrated into a target genome by nuclease-assisted vector integration, such that guide RNAs direct removal of the ITRs prior to transgene integration. In some aspects, the disclosure provides isolated nucleic acids that comprise an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more gene editing molecules (e.g., Cas9). The transgene may also comprise a region encoding, for example, a miRNA binding site, and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAVS, AAV6, AAV6.2, AAV7, AAV8, AAV9, AAV10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

In some embodiments, an isolated nucleic acid further encodes an mRNA encoding a protein. Generally, for nucleic acids encoding a protein, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931.; and Klump, H et al., Gene Therapy, 2001; 8: 811-817).

In some embodiments, the isolated nucleic acids described herein further comprise an expression cassette or sequence that is further engineered to express an mRNA encoding a protein. For example, an isolated nucleic acid can further comprise a therapeutic protein or a reporter protein. Reporter sequences that may be provided in an isolated nucleic acid include, without limitation, mCherry, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an isolated nucleic acid.

In some embodiments, the isolated nucleic acids described herein further comprise a therapeutic protein. Such therapeutic proteins may be useful for preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10, IL-11, IL-12, IL-13, IL-14, IL-15, IL-16 IL-17, and IL-18. In some embodiments, a therapeutic protein compensates for aberrant expression (e.g., over-expression or reduced expression relative to a normal cell) or aberrant function (e.g., increased activity or reduced activity relative to a normal cell), of an endogenous protein.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter. In some embodiments, a promoter is a chicken beta-actin (CBA) promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart cell-specific gene expression capabilities. In some cases, the cell -specific regulatory sequences bind cell-specific transcription factors that induce transcription in a cell specific manner. Such cell-specific regulatory sequences (e.g., promoters, enhancers, etc..) are known in the art.

“Homology” refers to the percent identity between two polynucleotides or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. When referring to a polypeptide, or fragment thereof, the term “substantial homology” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. In some cases, highly conserved may refer to 100% identity. Identity is readily determined by one of skill in the art by, for example, the use of algorithms and computer programs known by those of skill in the art.

As described herein, alignments between sequences of nucleic acids or polypeptides are performed using any of a variety of publicly or commercially available Multiple Sequence Alignment Programs, such as “Clustal W”, accessible through Web Servers on the internet.

Alternatively, Vector NTI utilities may also be used. There are also a number of algorithms known in the art which can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program which provides at least the level of identity or alignment as that provided by the referenced algorithms and programs. Alignments may be used to identify corresponding amino acids between two proteins or peptides. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid.

Alternatively for nucleic acids, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

Target Nucleic Acid Sequence As used herein, a “target nucleic acid sequence” generally refers to any genomic locus or site that is targeted by a gRNA and/or nuclease for gene editing (e.g., insertion of a transgene without any viral nucleic acid sequence (e.g., AAV ITR sequence) into a target locus). In some embodiments, a target nucleic acid sequence is in a host cell or a subject. In some embodiments, a target nucleic acid sequence is located within, adjacent to, or near a gene of interest within a genome. In some embodiments, a target nucleic acid is present in a safe harbor genome locus.

As used herein, the term “safe harbor locus” generally refers to any locus or site of genomic DNA that can accommodate a genetic insertion into said locus or site without adversely affecting the cell (e.g., reducing the reproductive fitness, or viability of the cell). In some embodiments, a safe harbor locus is located within or external to a gene. In some embodiments, a safe harbor locus is a site of genomic DNA that is transcriptionally silent. In some embodiments, a safe harbor locus is a site of genomic DNA that is highly methylated. In some embodiments, a safe harbor locus is a adeno-associated virus site 1 (AAVS1), chemokine (C-C motif) receptor 5 (CCR5) gene, human ortholog of the mouse Rosa26 locus, ALB, Angptl3, ApoC3, ASGR2, CCR5, FIX (F9), G6PC, Gys2, HGD, Lp(a), Pcsk9, Serpinal, TF, or TTR genome locus. In some embodiments, a safe harbor locus is as described by Papapetrou, E.P. and Schambach, A. “Gene Insertion Into Genomic Safe Harbors for Human Gene Therapy” Mol Ther. 2016 April; 24(4): 678-684.

In some embodiments, a target nucleic acid sequence, after delivery of AAV-NAVI constructs described herein, comprises an inserted gene. In some embodiments, an inserted gene may encode a protein (e.g., a reporter protein or a therapeutic protein)

### Adeno-Associated Virus (AAV)

In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV8, AAVrh8, AAV9, and AAV10. In some embodiments, an AAV capsid protein is of a serotype derived from a non-human primate, for example AAVrh8 serotype. In some embodiments, the AAV capsid protein is of a serotype that has tropism for the eye of a subject, for example an AAV (e.g., AAV5, AAV6, AAV6.2, AAV7, AAV8, AAV9, AAVrh.8, AAVrh.10, AAVrh.39 and AAVrh.43) that transduces ocular cells of a subject more efficiently than other vectors. In some embodiments, an AAV capsid protein is of an AAV8 serotype or an AAV5 serotype. In some embodiments, an AAV capsid protein is an AAV9 capsid protein.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a gene editing molecule (e.g., Cas9), an rAAV, and/or a target nucleic acid. In some embodiments, the instant disclosure relates to a composition comprising the host cell as described herein. In some embodiments, the composition comprising the host cell as described herein further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

### Methods for Delivery of AAV-NAVI Constructs and Inserting a Gene at Target Locus

Methods for delivering an isolated nucleic acid are provided herein. The methods typically involve administering to cells an effective amount of a rAAV comprising an isolated nucleic acid described herein. In some embodiments, an effective amount of a rAAV may be co-administered or introduced with a nuclease into a cell.

An “effective amount” of a rAAV is an amount sufficient to infect a sufficient number of cells of a population of cells. An effective amount of a rAAV may be an amount sufficient to induce gene editing in the cell, e.g., to insert a gene or transgene without any viral nucleic acid sequence (e.g., AAV ITR sequence) into a target locus of a genome. The effective amount will depend on a variety of factors such as, for example, the species, age, source of the cell and may thus vary among different cell types.

An effective amount may also depend on the rAAV used. The invention is based, in part on the recognition that rAAV comprising capsid proteins having a particular serotype (e.g., AAV1, AAV5, AAV6, AAV6.2, AAV7, AAV8, AAV9, AAVrh.8, AAVrh.10, AAVrh.39, and AAVrh.43) mediate more efficient transduction of cells of a pre-implantation embryo than rAAV comprising capsid proteins having a different serotype. Thus in some embodiments, the rAAV comprises a capsid protein of an AAV serotype selected from the group consisting of: AAV2, AAV5, AAV6, AAV6.2, AAV7, AAV8, AAV9, AAVrh.8, AAVrh.10, AAVrh.39, and AAVrh.43. In some embodiments, the rAAV comprises a capsid protein of AAV6 serotype. In some embodiments, the capsid protein is AAV6 capsid protein.

In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject. In some cases, multiple doses of a rAAV are administered.

In some aspects, the disclosure provides a method for inserting a gene into a target locus of a genome (e.g., an insertion of a transgene without any viral nucleic acid sequence (e.g., AAV ITR sequence) into a target locus), the method comprising: administering to a cell (i) an effective amount of an isolated nucleic acid, wherein the isolated nucleic acid comprises an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs; or (ii) an effective amount of a rAAV, wherein the rAAV comprises an isolated nucleic acid comprising an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs, and at least one AAV capsid protein. In some embodiments, the methods further comprise administering a nuclease (e.g., a plasmid or viral vector encoding a nuclease) to the cell.

In some embodiments, the cell is located within a subject (e.g., a mammalian subject, e.g., a human, primate, mouse, or rat subject). In some embodiments, the cell is in vitro or ex vivo.

The rAAVs may be delivered to a subject in compositions according to any appropriate methods known in the art. The rAAV, preferably suspended in a physiologically compatible carrier (i.e., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments, a host animal does not include a human.

Delivery of the rAAVs to a mammalian subject includes, but is not limited to, transplantation of a cell transduced with rAAVs into the subject and injection of rAAVs into the subject. In some embodiments, the delivery of the rAAVs to the mammalian subject comprises combinations of administration methods (e.g., transplantation and injection). In some embodiments, administration by injection may be done using vein (e.g., tail or facial vein injection), intramuscular, or peritoneal injection.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect cells and to provide sufficient levels of gene transfer and expression without undue adverse effects. Examples of pharmaceutically acceptable routes of administration include, but are not limited to, contacting rAAVs with a cell in vitro and contacting rAAVs with a cell in vivo. Routes of administration to a subject may be combined, if desired.

The dose of rAAV virions required to achieve a particular “gene editing effect,” e.g., the units of dose in genome copies/per kilogram of body weight (GC/kg), will vary based on several factors including, but not limited to: the route of rAAV virion administration, the level of gene or RNA expression required to achieve a gene editing effect, the specific gene being edited, and the stability of the gene or RNA product. One of skill in the art can readily determine a rAAV virion dose range to induce a gene editing effect in an embryonic cell based on the aforementioned factors, as well as other factors.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year). In some embodiments, a dose of rAAV is administered to a subject no more than once per two calendar years (e.g., 730 days or 731 days in a leap year). In some embodiments, a dose of rAAV is administered to a subject no more than once per three calendar years (e.g., 1095 days or 1096 days in a leap year).

In some embodiments, rAAV compositions are formulated to reduce aggregation of AAV particles in the composition, particularly where high rAAV concentrations are present (e.g., ˜1013 GC/ml or more). Appropriate methods for reducing aggregation of may be used, including, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, rAAVs in suitably formulated pharmaceutical compositions disclosed herein are delivered directly to a cell. However, in certain circumstances it may be desirable to separately or in addition deliver the rAAV-based therapeutic constructs via another route, e.g., subcutaneously, topically, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a suitable sterile aqueous medium may be employed. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Cells

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In some embodiments, a cell is in vitro or ex vivo. In some embodiments, a cell is maintained in culture media. In some embodiments, a cell is a liver, spleen, intestinal, epithelial, muscle, neural, brain, or reproductive cell.

In some embodiments, a cell is characterized by aberrant expression (e.g., over-expression or reduced expression relative to a normal cell) or aberrant function (e.g., increased activity or reduced activity relative to a normal cell), of a protein or gene. In some embodiments, a cell is characterized by aberrant expression of a protein or gene if said protein or gene is expressed in the cell at least 2-fold, 3-fold, 4-fold, 5-fold, 10-fold, 15-fold, 20-fold, 25-fold higher than a control cell (e.g., a healthy cell). In some embodiments, a cell is characterized by aberrant expression of a protein or gene if said protein or gene is expressed in the cell at least 2-fold, 3-fold, 4-fold, 5-fold, 10-fold, 15-fold, 20-fold, 25-fold lower than a control cell (e.g., a healthy cell). In some embodiments, a cell is characterized by aberrant function of a protein or gene if said protein or gene is functioning in the cell at functional levels that are at least 2-fold, 3-fold, 4-fold, 5-fold, 10-fold, 15-fold, 20-fold, 25-fold higher than a control cell (e.g., a healthy cell). In some embodiments, a cell is characterized by aberrant function of a protein or gene if said protein or gene is functioning in the cell at functional levels that are at least 2-fold, 3-fold, 4-fold, 5-fold, 10-fold, 15-fold, 20-fold, 25-fold lower than a control cell (e.g., a healthy cell). In some embodiments, aberrant expression or function of a protein or gene results from a genetic mutation of said protein or gene. In some embodiments, aberrant expression or function of a protein or gene is the result or cause of a disease.

### Subject

In some embodiments, the cell is located within a subject (e.g., a mammalian subject, e.g., a human, primate, mouse, or rat subject). In some embodiments, the cell is in vitro or ex vivo. In some embodiments, a subject is a host animal. In some embodiments, a subject is a mammalian subject. In some embodiments, a subject is a a human, mouse, rat, cat, dog, sheep, rabbit, horse, cow, goat, pig, guinea pig, hamster, chicken, turkey, or a non-human primate (e.g., Macaque). In some embodiments, a subject is a human subject.

In some embodiments, a subject is has or is suspected of having a disease associated with aberrant expression and/or aberrant function of a gene or protein. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppessor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing an isolated recombinant Adeno-Associated Virus (rAAV) for gene editing in a cell of a pre-implantation embryo, comprising at least one container housing a rAAV vector, wherein the rAAV comprises at least one capsid protein, and a nucleic acid comprising a promoter operably linked to a transgene encoding a gene editing molecule, at least one container housing a rAAV packaging component, and instructions for constructing and packaging the rAAV.

In some embodiments, a kit may comprise (i) an isolated nucleic acid as described herein (e.g., comprising at least one transgene flanked by inverted terminal repeats (ITRs), wherein the transgene is configured to be integrated into a target genome by nuclease-assisted vector integration, such that guide RNAs direct removal of the ITRs prior to transgene integration; or comprising an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs); (ii) a rAAV as described herein; and/or (iii) a nuclease.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### EXAMPLES

### Example 1: Nuclease-Mediated Viral Integration (NAVI) Improves the Safety and Efficacy of rAAV-Mediated Transgene Integration

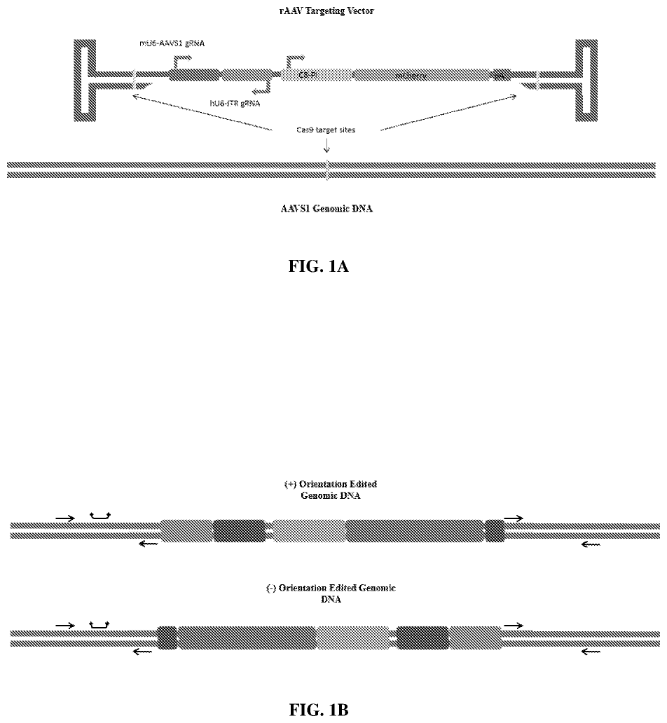

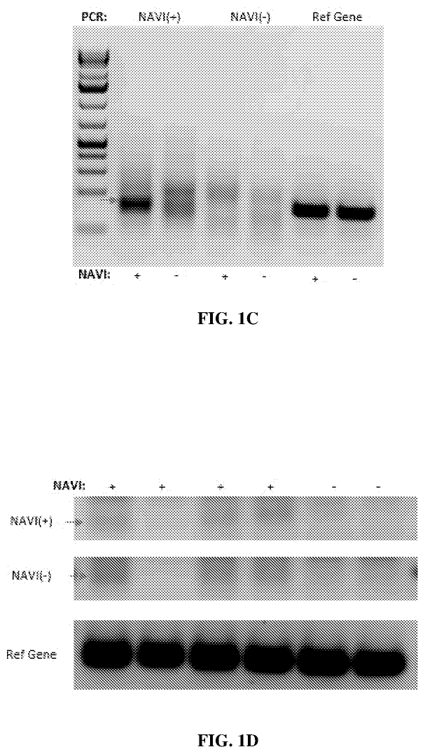

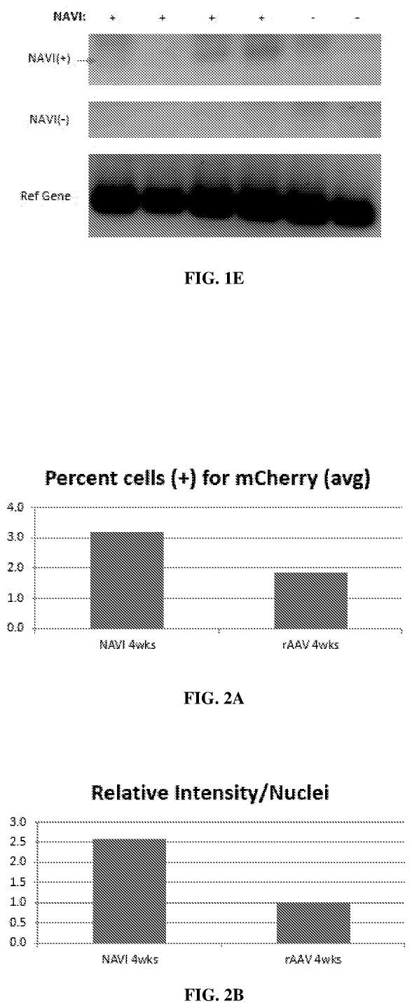

Guide sequences for production of guide-RNA (gRNA) targeting the wild-type ITRs of AAV were designed and evaluated for rAAV-NAVI (Table 1). Three gRNAs at the distal end of the ITR that may be utilized for both Streptococcus pyogenes (Sp) and Staphylococcus aureus (Sa) Cas9 gene editing were identified. SpCas9 recognizes ˜20 bases upstream of a NGG proto-spacer adjacent motif (PAM), while SaCas9 recognizes PAMs of the NNGRRT (SEQ ID NO: 1) and NNGRR (SEQ ID NO: 2) types. The three selected guides have NGGRRT sequences flanking the target region and are suitable for both SpCas9 and SaCas9 gene editing therapeutics. Examples of rAAV-NAVI vectors are depicted in FIGS. 1A and 1B.

TABLE 1
Cas9 guide target sites of the ITR

| | | | | | |
|---|---|---|---|---|---|
| | | | | Specificity | Efficiency |
| Position | Strand | Sequence | PAM | Score | Score |

SpCas9

| | | | | | |
|---|---|---|---|---|---|
| 20 | (+) | GCGCGCTCGCTCGCTCACTG | AGG | 67.5 | 43.2 |
| | | (SEQ ID NO: 3) | (SEQ ID | | |
| | | | NO: 32) | | |
| 28 | (+) | GCTCGCTCACTGAGGCCGCC | CGG | 63.7 | 41.5 |
| | | (SEQ ID NO: 4) | (SEQ ID | | |
| | | | NO: 33) | | |
| 29 | (+) | CTCGCTCACTGAGGCGCCC | GGG | 55.2 | 4.7 |
| | | (SEQ ID NO: 5) | (SEQ ID | | |
| | | | NO: 34) | | |
| 32 | (−) | ACGCCCGGGCTTTGCCCGGG | CGG | 76.8 | 7.3 |
| | | (SEQ ID NO: 6) | (SEQ ID | | |
| | | | NO: 35) | | |
| 35 | (−) | CCGACGCCCGGGCTTTGCCC | GGG | 67.2 | 10.2 |
| | | (SEQ ID NO: 7) | (SEQ ID | | |
| | | | NO: 36) | | |
| 36 | (−) | CCCGACGCCCGGGCTTTGCC | CGG | 69.3 | 7.6 |
| | | (SEQ ID NO: 8) | (SEQ ID | | |
| | | | NO: 37) | | |
| 39 | (+) | GAGGCCGCCCGGGCAAAGCC | CGG | 61.4 | 13.1 |
| | | (SEQ ID NO: 9) | (SEQ ID | | |
| | | | NO: 38) | | |
| 40 | (+) | AGGCCGCCCGGGCAAAGCCC | GGG | 57.9 | 26.7 |
| | | (SEQ ID NO: 10) | (SEQ ID | | |
| | | | NO: 39) | | |
| 46 | (+) | CCCGGGCAAAGCCCGGGCGT | CGT | 76.8 | 5.0 |
| | | (SEQ ID NO: 11) | (SEQ ID | | |
| | | | NO: 40) | | |
| 46 | (−) | CCAAAGGTCGCCCGACGCCC | GGG | 89.8 | 19.7 |
| | | (SEQ ID NO: 12) | (SEQ ID | | |
| | | | NO: 41) | | |
| 47 | (+) | CCGGGCAAAGCCCGGGCGTC | GGG | 80.0 | 0.3 |
| | | (SEQ ID NO: 13) | (SEQ ID | | |
| | | | NO: 42) | | |
| 47 | (−) | ACCAAAGGTCGCCCGACGCC | CGG | 92.5 | 10.9 |
| | | (SEQ ID NO: 14) | (SEQ ID | | |
| | | | NO: 43) | | |
| 57 | (+) | CCCGGGCGTCGGGCGACCTT | TGG | 91.8 | 3.6 |
| | | (SEQ ID NO: 15) | (SEQ ID | | |
| | | | NO: 44) | | |
| 62 | (−) | CACTAGGCCGGGCGACCAA | AGG | 83.4 | 14.0 |
| | | (SEQ ID NO: 16) | (SEQ ID | | |
| | | | NO: 45) | | |
| 65 | (+) | TCGGGCGACCTTTGGTCGCC | CGG | 94.5 | 3.3 |
| | | (SEQ ID NO: 17) | (SEQ ID | | |
| | | | NO: 46) | | |
| 72 | (−) | CTCGCTCGCTCACTGAGGCC | GGG | 39.3 | 10.6 |
| | | (SEQ ID NO: 18) | (SEQ ID | | |
| | | | NO: 47) | | |
| 73 | (−) | GCTCGCTCGCTCACTGAGGC | CGG | 27.6 | 25.5 |
| | | (SEQ ID NO: 19) | (SEQ ID | | |
| | | | NO: 48) | | |
| 77 | (−) | GCGCGCTCGCTCGCTCACTG | AGG | 67.5 | 50.0 |
| | | (SEQ ID NO: 20) | (SEQ ID | | |
| | | | NO: 49) | | |
| 95 | (+) | GAGCGAGCGAGCGCGCAGAG | AGG | 39.3 | 23.8 |
| | | (SEQ ID NO: 21) | (SEQ ID | | |
| | | | NO: 50) | | |
| 96 | (+) | AGCGAGCGAGCGCGCAGAGA | GGG | 67.1 | 3.7 |
| | | (SEQ ID NO: 22) | (SEQ ID | | |
| | | | NO: 51) | | |
| 101 | (+) | GCGAGCGCGCAGAGAGGGA | TGG | 33.3 | 19.8 |
| | | G (SEQ ID NO: 23) | (SEQ ID | | |
| | | | NO: 52) | | |
| 113 | (−) | GGAACCCCTAGTGATGGAGT | TGG | 54.5 | 12.0 |
| | | (SEQ ID NO: 24) | (SEQ ID | | |
| | | | NO: 53) | | |
| 118 | (+) | GAGTGGCCAACTCCATCACT | AGG | 58.4 | 13.4 |
| | | (SEQ ID NO: 25) | (SEQ ID | | |
| | | | NO: 54) | | |
| 119 | (+) | AGTGGCCAACTCCATCACTA | GGG | 58.7 | 45.0 |
| | | (SEQ ID NO: 26) | (SEQ ID | | |
| | | | NO: 55) | | |
| 119 | (−) | CTACAAGGAACCCCTAGTGA | TGG | 70.0 | 44.0 |
| | | (SEQ ID NO: 27) | (SEQ ID | | |
| | | | NO: 56) | | |
| 120 | (+) | GTGGCCAACTCCATCACTAG | GGG | 72.0 | 18.1 |
| | | (SEQ ID NO: 28) | (SEQ ID | | |
| | | | NO: 57) | | |

SaCas9

| | | | | | |
|---|---|---|---|---|---|
| 96 | (+) | GAGCGAGCGAGCGCGCAGAG | GGGAGT | 73.6 | 3.7 |
| | | A (SEQ ID NO: 29) | (SEQ ID | | |
| | | | NO: 58) | | |
| 118 | (+) | GGAGTGGCCAACTCCATCAC | AGGGGT | 72.3 | 13.4 |
| | | T (SEQ ID NO: 30) | (SEQ ID | | |
| | | | NO: 59) | | |
| 119 | (−) | ACTACAAGGAACCCCTAGTG | TGGAGT | 82.2 | 44.0 |
| | | A (SEQ ID NO: 31) | (SEQ ID | | |
| | | | NO: 60) | | |

Experiments of rAAV-NAVI were carried out in neonatal mice and analyzed. FIG. 1C shows a representative end-point PCR detection of vector integration from mouse liver tissue 4 weeks after neonatal infection with rAAV-NAVI virus (10¹¹ viral genome copies/pup, facial vein) with preferential vector orientation. Analyses of heart (FIG. 1D) and muscle (FIG. 1E) genomic DNA indicate tissue-specific patterns of integration achieved by rAAV-NAVI.

Integration was confirmed directly by Sanger sequencing of select sample amplicons. Sequenced amplicons were aligned to either NAVI (+) or (−) integration maps (Table 2, below). As NAVI proceeds via error-prone repair pathways, insertion and deletion events (indels) can be used to provide estimates of total numbers of edited alleles, when compared to genomic DNA. None of the sequenced sites of integration that were sequenced revealed any indication of viral vector ITR or other cleavage artifacts.

TABLE 2
rAAV-NAVI integration detection by sequencing

| | | | | # | # |
|---|---|---|---|---|---|
| Mouse | NAVI (+/−) | Mapped | Clones | Mapped | Unique |
| 1-5 | + | B, D, E, F, I, J | 12 | 7 | 6 |
| 1-5 | − | F, G | 12 | 2 | 2 |
| 1-6 | + | A, B, C, D, E, G | 12 | 9 | 5 |
| 1-6 | − | B, E | 12 | 2 | 2 |
| 1-7 | + | B, C, D, E, F, G | 12 | 11 | 9 |
| 1-7 | − | A, B, E, F, G, I | 12 | 7 | 6 |
| 1-8 | + | B, C, E, F, G, H | 12 | 10 | 9 |
| 1-8 | − | | 10 | 0 | 0 |

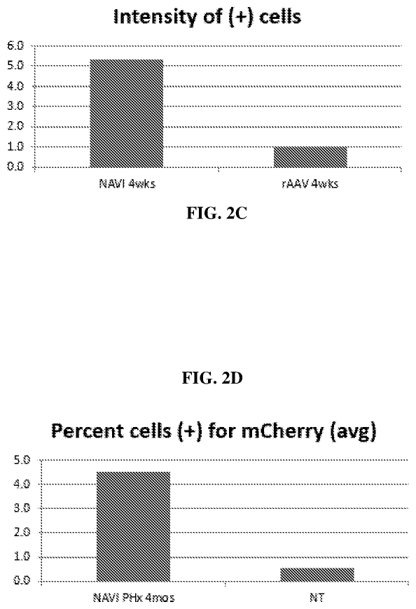

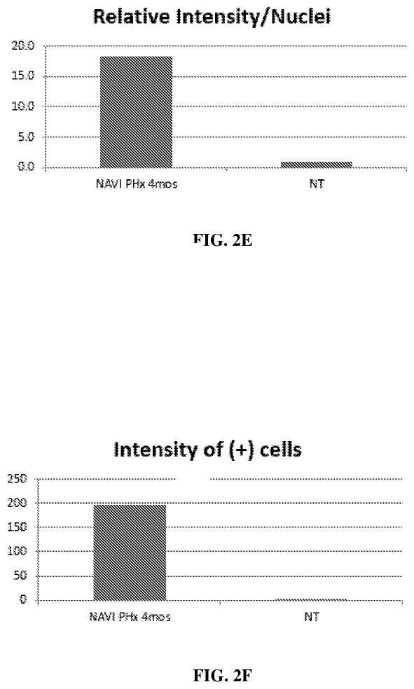

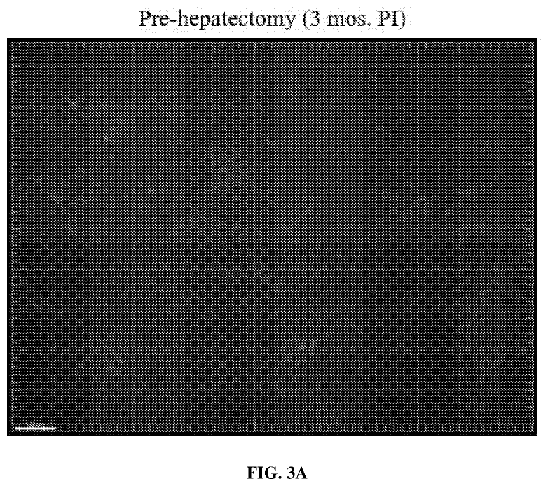

Quantification and analysis by fluorescence microscopy for rAAV-NAVI demonstrates robust patterns of transgene expression, exceeding that of standard transient transduction via (rAAV). Four weeks following infection, greater than 3% of liver cells still highly express the mCherry reporter gene (FIG. 2A), representing ˜2-fold increase above transient rAAV infection. Additionally, overall transgene expression within rAAV-NAVI samples was approximately 150% higher on average (FIG. 2B). As transient rAAV transgene expression may still occur by rAAV-NAVI delivery, the total number of cells expressing the reporter gene and average intensity is not entirely reflective of NAVI-dependent expression, as episomal expression of rAAV may persist over long periods. However, since NAVI expression occurs in a genomic context, transcriptional regulation may be altered, as evidence by higher levels of reporter signal from positive cells (FIG. 2C).

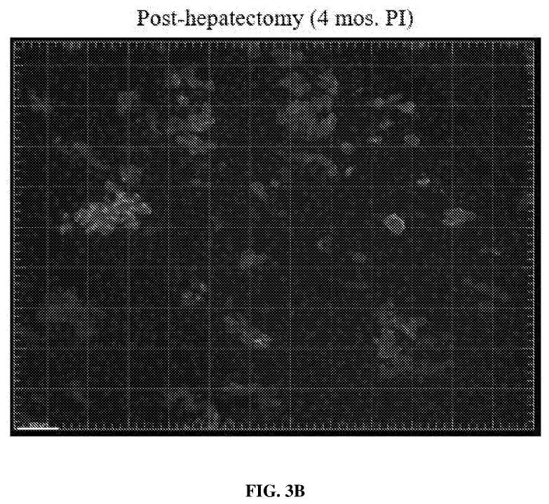

To better estimate the potential of rAAV-NAVI for sustained expression in rapidly-dividing tissues over a longer-term, select mice underwent partial hepatectomy at 3 months. Following a 4-week recovery for compensatory liver tissue growth, tissue samples were analyzed by microscopy as before (FIGS. 2D-2F). Remarkably, over 4% of cells maintained expression in rAAV-NAVI treated liver tissue. Furthermore, both average and positive cell-specific reporter signal intensities increased dramatically, as compared to 4-week post-infection samples (FIGS. 3A-3B).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An isolated nucleic acid comprising at least one transgene flanked by inverted terminal repeats (ITRs), wherein the transgene is configured to be integrated into a target genome by nuclease-assisted vector integration, such that guide RNAs direct removal of the ITRs prior to transgene integration.

2. An isolated nucleic acid comprising an expression cassette engineered to express a first guide RNA (gRNA), wherein the expression cassette is flanked by inverted terminal repeats (ITRs), wherein the gRNA targets (e.g., hybridizes with) a nucleic acid sequence located adjacent to or within the nucleic acid sequence encoding the ITRs.

3. The isolated nucleic acid of claim 2, wherein the gRNA comprises a NNGRRT (SEQ ID NO: 1) or a NNGRR (SEQ ID NO: 2) sequence, optionally wherein the gRNA comprises a sequence set forth in Table 1.

4. The isolated nucleic acid of claim 2 or 3, wherein the expression cassette is further engineered to express a second gRNA that targets (e.g. hybridizes with) a target nucleic acid sequence that is not present in the isolated nucleic acid.

5. The isolated nucleic acid of claim 4, wherein the target nucleic acid sequence is located in a host cell.

6. The isolated nucleic acid of claim 4 or 5, wherein the target nucleic acid sequence is present in a safe harbor genome locus, optionally wherein the safe harbor genome locus is AAVS1 genome locus.

7. The isolated nucleic acid of any one of claims 2 to 6, wherein the expression cassette is further engineered to express an mRNA encoding a protein, optionally wherein the protein is a reporter protein or a therapeutic protein.

8. A recombinant adeno-associated virus (rAAV) comprising:
(i) the isolated nucleic acid of any one of claims 1 to 7; and
(ii) at least one AAV capsid protein.

9. The rAAV of claim 8, wherein the at least one capsid protein is AAV1, AAV2, AAV3, AAV4, AAV5, AAV6, AAV7, AAV8, AAV9 capsid protein.

10. The rAAV of claim 9 or 10, wherein the at least one capsid protein is an AAV9 capsid protein.

11. A composition comprising:
(i) the rAAV of any one of claims 8 to 10; and
(ii) a nuclease.

12. The composition of claim 11, wherein the nuclease is a Transcription Activator-like Effector Nuclease (TALEN), Zinc-Finger Nuclease (ZFN), engineered meganuclease, re-engineered homing endonuclease, or a Cas-family nuclease.

13. The composition of claim 11 or 12, wherein the nuclease is a Cas-family nuclease, optionally wherein the Cas-family nuclease is a Cas9 or Cas? nuclease.

14. The composition of claim 12 or 13, wherein the Cas-family nuclease is a Streptococcus pyogenes (Sp) or a Staphylococcus aureus (Sa) Cas9 nuclease.

15. The composition of any one of claims 11 to 14, wherein the nuclease is encoded by a plasmid or a viral vector, optionally wherein the viral vector is an rAAV vector.

16. A method for inserting a gene into a target locus of a genome, the method comprising introducing into a cell:
(i) the isolated nucleic acid of any one of claims 1 to 7, or the rAAV of any one of claims 8 to 10, and a nuclease; or,
(ii) the composition of any one of claims 11 to 15.

17. The method of claim 16, wherein the nuclease is a Transcription Activator-like Effector Nuclease (TALEN), Zinc-Finger Nuclease (ZFN), engineered meganuclease, re-engineered homing endonuclease, or a Cas-family nuclease.

18. The method of claim 16 or 17, wherein the nuclease is a Cas-family nuclease, optionally wherein the Cas-family nuclease is a Cas9 or Cas? nuclease.

19. The method of claim 17 or 18, wherein the Cas-family nuclease is a Streptococcus pyogenes (Sp) or a Staphylococcus aureus (Sa) Cas9 nuclease.

20. The method of any one of claims 16 to 19, wherein the nuclease is encoded by a plasmid or a viral vector, optionally wherein the viral vector is an rAAV vector.

21. The method of any one of claims 16 to 20, wherein the introducing results in insertion of the transgene without any viral nucleic acid sequence (e.g., AAV ITR sequence) into the target locus.

22. The method of any one of claims 16 to 21, wherein the target locus is a safe harbor genome locus, optionally wherein the safe harbor genome locus is AAVS1 genome locus.

23. The method of any one of claims 16 to 22, wherein the cell is in a subject, optionally wherein the subject is a human.

24. The method of any one of claims 16 to 22, wherein the cell is in vitro or ex vivo.

25. The method of any one of claims 16 to 24, wherein the cell is characterized by aberrant expression (e.g., over-expression or reduced expression relative to a normal cell) or aberrant function (e.g., increased activity or reduced activity relative to a normal cell), of a protein.
